(19) United States
(12) Patent Application Publication
(10) Pub. No.: US 2024/0174846 A1
(43) Pub. Date: May 30, 2024

(54) AMINOALKYL PIPERAZINE (POLY) PYROPHOSPHATES AND FLAME RETARDED RESIN COMPOSITIONS MADE THEREFROM

(71) Applicant: ICL-IP America Inc., Tarrytown, NY (US)

(72) Inventors: Meng Zhang, Chappaqua, NY (US); Gerald Alessio, Norwood, NJ (US); George Schlinck, New York, NY (US); Sergei Levchik, Croton on Hudson, NY (US); Andrew Piotrowski, Yorktown Heights, NY (US)

(21) Appl. No.: 17/986,138

(22) Filed: Nov. 14, 2022

(51) Int. Cl. C08L 23/12

(52) U.S. Cl. CPC C08L 23/12

(57) ABSTRACT

Aminoalkyl piperazine (poly) pyrophosphates as effective flame retardants and thermoplastic polymer resins containing such flame retardants. These flame retarded resins are particularly useful in electrical and electronic components, such as casing or coverings for metal wires or cables.

### FIELD OF THE INVENTION

The present invention relates to flame retardant aminoalkyl piperazine (poly)pyrophosphates having high thermal stability and low melting/softening points. The invention also relates to a method of producing the same and resin compositions, more specifically flame retarded resin compositions, which can be used in the manufacture of electrical and electronic components, such as casings for electronic equipment and insulated cables and wires.

### BACKGROUND OF THE INVENTION

Flame retardancy can be an important property of articles made, for example, from thermoplastic resins, such as polyolefins. Flame retardants are chemicals that resist ignition and slow down the spread of fire. They are used in, for example, thermoplastics, textiles, and coatings. Typically, flame retardants are halogenated (i.e., brominated), organophosphorus or inorganic compounds. Because polyolefins are very flammable even the most efficient halogenated flame retardants usually require about 30-35% loading in the resin along with 8-10% antimony trioxide synergist.

Organophosphorus and inorganic flame retardants, tend to be even less efficient than halogenated flame retardants. Generally, these flame retardants require high loading (i.e., doses/volumes) which reduces efficacy. Such high doses may compromise the mechanical properties of the article they are intended to protect, thereby increasing susceptibility to failure of polyolefins and other materials to which these types of flame retardants are applied. The high percentage can compromise the structural integrity of the article and can cause the properties of the final product to deteriorate. Higher loading of flame retardant also creates problems during processing/extruding as solid flame retardants can be incompatible with the molten polymers during processing.

Few intumescent flame retardants based on phosphorous and nitrogen-based compounds are available that can impart flame retardancy to polyolefins. Examples include, ammonium polyphosphates, piperazine (poly)pyrophosphates, melamine (poly)pyrophosphates, ethylenediamine phosphate, and melamine phosphates. The loading percentage of intumescent flame retardants in polyolefin formulations has varied from 20-40 wt. %, more preferably from 28 to 35 wt. % of the total weight of the resin. Such available intumescent flame retardants are of particulate nature and lack practical melting points, such as at or below temperatures typically encountered during resin extrusion. For example, such flame retardants typically have a melting point above 250° C. or do not at all melt before decomposition. When such materials are used in extruder applications, the non-meltable nature of the particles can lead to agglomeration and decomposition due to high shear forces in extruders.

Therefore, it is desirable to provide flame retardants that exhibit good flame retardancy and physical properties and overcome drawbacks of the prior art.

### SUMMARY OF THE INVENTION

Generally speaking, in accordance with the invention, improved flame retardant compounds, resins including the same and methods of preparation are provided. Preferred flame retardants in accordance with the invention have the following general formula (I):

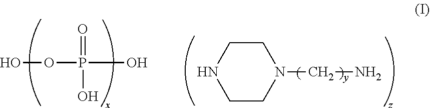

where x≥2; y is from 2 to 6; and z≥1; and x≥z, provided that when z is 1, x is ≥2. Typically, x is from 2-6, y is from 2-6, and z is from 1-6. Preferably x is from 2-4, y is from 2-4, and z is from 1-4. Most preferably, x is 2, y is 2, and z is 1.

In a preferred embodiment of the invention, y is 2. In another preferred embodiment of the invention, x is 2. In other preferred embodiments, both x and y are 2. Preferred compounds within the scope of Formula I include aminoethyl piperazine pyrophosphate (x=2, y=2, and z=1), and aminoethyl piperazine polyphosphate (x>2, y=2, and z≥1.

Preferred methods of preparing compounds of Formula I include reacting aminoalky piperazine, preferably aminoethyl piperazine, with polyphosphoric acid with heat and agitation. For purposes of combining with resins, the reaction product can be ground into a powder for ease of use. The ground powder can be surface coated, for example, with a silane or a siloxane-based additive, for example, triethoxy(octyl) silane, (3-aminopropyl)triethoxysilane, vinyltriethoxysilane, polyoctahedral silsesquioxanes (“POSS”), or other suitable coating materials, and mixed with melamine pyrophosphate or other nitrogen and phosphorus containing flame retardants.

In another embodiment of the invention, flame retardant compounds of Formula I can be prepared by mixing piperazine and aminoalkyl piperazine (preferably 50/50) with phosphoric acid to prepare a mixture of piperazine and aminoalkyl piperazine pyrophosphate. This combination is mixed thoroughly and dried. The dried mixture is exposed to heat and agitation to permit the reaction to proceed to obtain a mixture of piperazine pyrophosphate and aminoalkyl piperazine pyrophosphate. As stated above, the reaction product can be ground and surface coated with 1% of a silane or siloxane at elevated temperature. The coated material can be blended with melamine pyrophosphate and can be combined with resin material, melted and extruded as desired.

In another embodiment of the invention, flame retardant resins in accordance with the invention can be prepared by mixing the aminoalkyl piperazine (poly) pyrophosphates of Formula I with polymeric resin formulations, preferably thermoplastic resin formulations, such as polyolefin containing resin formulations. Preferred flame retardant resins in accordance with the invention contain at least 10 wt. %, preferably at least 15 wt. % aminoalkyl piperazine (poly) pyrophosphate of the total weight of the resin material. The combined formulation can be melted and extruded as desired, such as to form molded electrical and electronic equipment components and insulating sheaths or coverings for metal wires or cables.

Flame retarded resin compositions containing aminoalkyl piperazine (poly) pyrophosphates in accordance with the invention are effectively free from particles and agglomerates that can interfere with extrusion processing. Upon testing, extruded products made therefrom achieve a V-0 fire resistance rating.

### BRIEF DESCRIPTION OF THE DRAWINGS

FIG. 1 is a 7× magnification photograph of: (a) a comparative example polypropylene film (Film A) made with a commercially available prior art melamine based ammonium polyphosphate flame retardant (Comparative Example 6), (b) a comparative example polypropylene film (Film B) made with a commercially available prior art piperazine pyrophosphate and melamine pyrophosphate flame retardant (Comparative Example 7), and (c) a 7× magnification photograph of a polypropylene film (Film C), made with a flame retardant formulation in accordance with the invention (Invention Example 4). The flame retarded Film C in accordance with the invention has no observable particles, in contrast with Films A and B, containing the prior art flame retardants, which exhibit particles.

The drawings are for purposes of illustration only and are not intended to be interpreted to limit the scope of the invention.

### DETAILED DESCRIPTION OF THE PREFERRED EMBODIMENTS

The present disclosure may be understood more readily by reference to the following detailed description of the disclosure taken in connection with the accompanying figures, which form a part of this disclosure. It is to be understood that this disclosure is not limited to the specific materials, methods, conditions or parameters described and/or shown herein, and that the terminology used herein is for the purpose of describing particular embodiments by way of example only and is not intended to be limiting of the claimed disclosure.

Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

The invention relates to improved flame retardants having high thermal stability and a low melting or softening point that are more compatible with polymer substrates, leading to less diminution of mechanical properties and increased ease of extrusion, as compared to existing flame retardants. Preferred flame retardants in accordance with the invention are capable of melting during extrusion processes at temperatures below about 250° C., preferably below about 225° C., most preferably at about 200° C., to avoid any agglomeration and occlusion issues that occur during extrusion with conventional flame retardants. In particular, the invention relates to aminoalkyl piperazine pyrophosphate and aminoalkyl piperazine polyphosphate flame retardants. The invention also relates to resin compositions, for example, thermoplastic resins containing flame retardants that can be used, for example, in the field electrical and electronic equipment, such as casings for electronic equipment and cable and wire coverings. Exemplary thermoplastic polymer resins include polyolefins, such as polyethylene, polypropylene, polycarbonates, polystyrene, polyamides, polyesters, and the like.

The flame retardants in accordance with the invention are based on phosphorous and nitrogen containing compounds that impart flame retardancy to the polymers with which they are mixed. In addition to the aminoalkyl piperazine pyrophosphates and aminoalkyl piperazine polyphosphates of the invention, flame retardant compositions in accordance with the invention can include other nitrogen and phosphorus containing compounds, such as e.g., ammonium polyphosphates, piperazine polyphosphates, ammonium pyrophosphates, piperazine pyrophosphates, melamine polyphosphates, melamine pyrophosphates, ethylenediamine phosphate, and their derivatives.

Furthermore, the flame retarded resin compositions may contain common suitable additives, such as antioxidants, anti-dripping agents, and the like. The total loading percentage of the flame retardants into resins, such as polyolefin-based resins, in accordance with the invention, can vary from 10 wt. % to 40 wt. %, preferably in the range from 28 wt. % to 35 wt. % of the total weight of the resin material. Furthermore, at least 10 wt. %, preferably at least 15 wt. % of the loading into the resin should be the aminoalkyl piperazine (poly) pyrophosphate in accordance with the invention.

Accordingly, the invention provides intumescent flame retardants that show good flame retardancy and physical properties and that are capable of more certain and definite melting or softening. This more definite melting or softening (below about 200° C., preferably at or below approximately 150° C.) leads to more intimate, homogeneous mixing prior to extrusion. This reduces agglomeration and occlusion issues. Examples of such intumescent flame retardants and resins containing the same are set forth below.

### EXAMPLES

The following Examples are provided for illustration purposes and are not intended to be interpreted as limiting the scope of the invention.

Preferred flame retardant compounds in accordance with the invention have the following general structure (I):

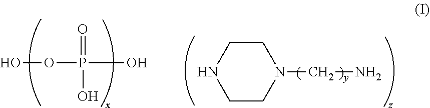

where x≥2; y is from 2 to 6; and z≥1; and x≥z, provided that when z is 1, x≥2.

### Example 1

### Synthesis of Flame Retardants of the Invention

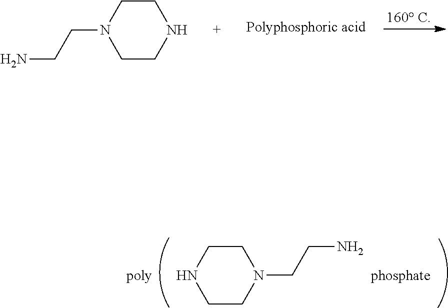

To an NH-1 mixer bowl (available from Laizhou Yuanda Factory), equipped with sigma blades, 437 grams of polyphosphoric acid was added at room temperature. The bowl cover was secured, and agitation was set at 1300 Hz, ˜50 rpm, and the jacket temperature set to 60° C. Through the sight glass port 334 grams of aminoethyl piperazine was fed. After the addition, the port was closed and a slight nitrogen purge was applied, batch temperature was 64° C., and jacket was 100° C. The reaction being exothermic caused an increase in batch temperature to 80° C. The batch was heated to 160° C., vacuum was applied (500 Ton) and held for 1 hour. The resulting compound was a dark yellow product which is in a molten state at reaction temperature. TGA of the product is 98% at 314° C., 95% at 329° C., and 90% at 345° C. This reaction product was confirmed by ³¹PNMR analysis. The product was taken out from the kneader, and ground using a Wiley Mill equipped with a 30-mesh screen. The material was passed through the mill twice. Following milling the product was surface coated with 1% of triethoxy(octyl) silane at 100° C. and held for 2 hours. The coated product was blended with melamine pyrophosphate (Aflammit PMN370, available from Thor Specialties LLC) at a 60/40 ratio for further evaluations.

### Example 2

### Synthesis of Flame Retardants of the Invention

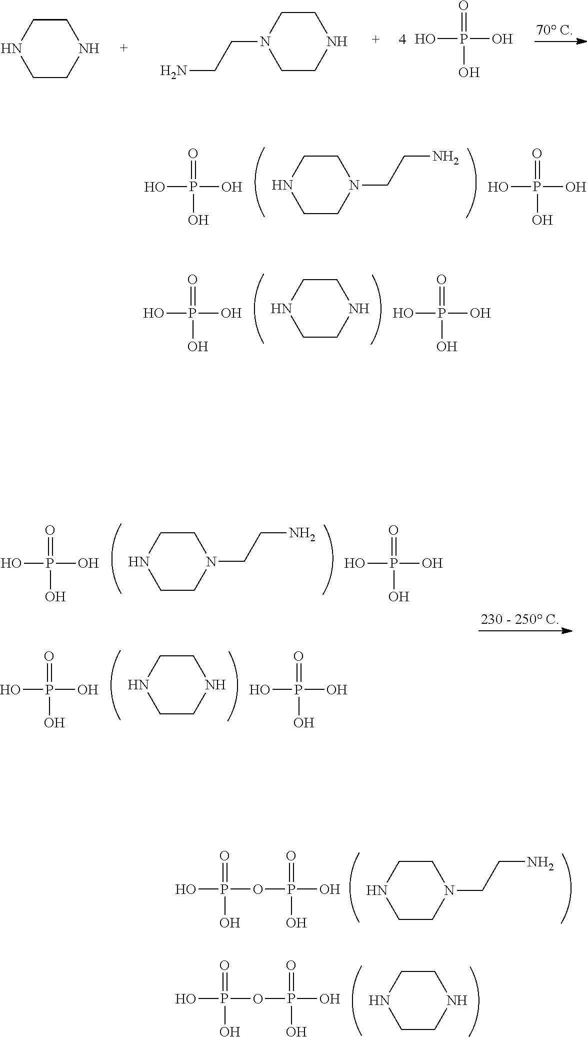

A 50/50 mixture of piperazine/aminoethyl piperazine diphosphate was initially prepared as follows. In a 2 L stainless steel bowl, piperazine (200 g, 2323 mmol) and aminoethyl piperazine (300 g, 2323 mmol) were mixed. 85 wt. % Phosphoric acid (536 g, 4646 mmol) was added dropwise over 2 hours. The mechanical mixer could not stir this mixture as it turned from a soft solid/liquid to a hard solid, so mixing was done directly by hand. The reaction was exothermal and water vapor and some piperazine vapor escaped from the open reaction bowl during hand mixing. The mixture formed a yellow “crumble” reaction product at the end. The product mixture was taken out of the reaction bowl and dried at 105° C. overnight. The same procedure was repeated one more time.

The dried reaction product mixture (about 1.5 kg) was collected and added into the NH-1 kneader bowl at RT with agitation. The jacket heaters were set at 80° C. At ˜80° C. the yellowish material started to soften, then within minutes melted. The temperature was then increased to 180° C. A slight nitrogen purge was started. Water vapor and a little free piperazine was carried out due to the purge. The temperature set point was increased to 230° C. and once reached held for 5 hours. The batch was allowed to cool and harden without agitation. ³¹P NMR analysis determined that the reaction was not sufficiently complete with about 30% of monophosphate left. Due to the high monophosphate content the batch was reheated to 250° C. and held for an addition 5.5 hours after which the batch was cooled and discharged. The reaction product was a dark yellow solid which is a molten state at reaction temperature. The product was not quite soluble in water. TGA of the product is 98% at 238° C., 95% at 295° C., and 90% at 327° C. The reaction product (a mixture of piperazine pyrophosphate and aminoethyl piperazine pyrophosphate) was confirmed by ³¹PNMR analysis. The product was ground using a Wiley Mill equipped with a 30-mesh screen. Following milling the product was surface coated with 1% of triethoxy(octyl) silane at 100° C. and held for 2 hours. The coated material was blended with melamine pyrophosphate (Aflammit PMN370 available from Thor Specialties LLC) at 60/40 ratio for further evaluations.

### Example 3

### Preparation of Flame Retardant Resins of the Invention and Comparisons

Polypropylene (PP) resin (1112 PP) was obtained from Pinnacle. Melamine pyrophosphate (Aflammit PMN370) was obtained from Thor. Irganox B225 was obtained from BASF as an antioxidant/thermal stabilizer. PTFE (FA-600) was obtained from Daikin as an anti-dripping agent.

Flame retardant compositions shown in Table 1 (below) were mixed with polypropylene resin, and the mixture was extruded using a small-scale Brabender extruder at 200° C. to obtain pellets. The resulting pellets from the extruder were dried at 75° C. for 24h, and then molded through an Arburg Injection Molder machine, wherein maximum temperature of its cylinder had been adjusted to 210° C., to provide test specimens. The test specimens were subjected to the tests for flammability and physical properties as described below.

### UL Flammability 94V Test:

UL-94 vertical burning test was measured with a specimen size of 127×12.7×1.6 mm according to the standard ASTM D 3801. The specimen was positioned vertically, and a burner was applied to the lower end of the specimen for 10 seconds. After 10 seconds, the flame was removed, and time required to self-extinguish (burning time) was recorded. As soon as the flame extinguished, the flame was immediately applied for another 10 seconds. Again, the burning time was recorded. The test allows the classification of the material at the 94 V-0, 94 V-1, and 94 V-2 levels depending on the burning time of the samples and whether they drop flaming drips.

### Tensile Properties Test:

The tensile tests were conducted according to the test standard ASTM D638 at ambient temperature. Specimens used were tensile bars 3.2 mm, and strain was applied at 2.0 in/min.

### IZOD Pendulum Impact Test:

The Izod notched impact measurements were performed according to ASTM D256. Specimens used were 3.2 mm bars, cut in half perpendicular to the length, then notched. Windage and friction loss was 0.03%.

### Heat Deflection Temperature (HDT) Test:

Heat deflection temperature was measured according to the test standard ASTM D648 by using 3.2 mm specimens. The test was conducted under 1.82 MPa, and the heat increase rate was 2.00° C. per minute.

TABLE 1
Flame retardant composition and physical properties.

| | Invention | Invention | Comparative | Comparative |
|---|---|---|---|---|
| | Example 4 | Example 5 | Example 6 | Example 7 |
| PP Resin | 69.6% * | 69.6 | 69.8% | 69.8% |
| Composition | | | | |
| Flame Retardant | 30.0% | | | |
| Formulation of | | | | |
| Example 1 | | | | |
| Flame Retardant | | 30.0% | | |
| Formulation of | | | | |
| Example 2 | | | | |
| Commercial | | | 30.0% | |
| Melamine based | | | | |
| ammonium | | | | |
| polyphosphate | | | | |
| Commercial | | | | 30.0% |
| Piperazine | | | | |
| pyrophosphate and | | | | |
| melamine | | | | |
| pyrophosphate | | | | |
| Daikin FA-600 | 0.2% | 0.2% | | |
| (PTFE) | | | | |
| Irganox B225 | 0.2% | 0.2% | 0.2% | 0.2% |
| UL-94V (1.6 mm), | V-0, no | V-0, no | V-0, non | V-0, no |
| dripping or not | dripping | dripping | flaming | dripping |
| | | | drips | |
| HDT (° C.) | 80.8 | 58.2 | 67.0 | 73.4 |
| IZOD Impact | 22.37 | 25.74 | 24.71 | 15.96 |
| (J/m) | | | | |
| Tensile Strength | 32.9 | 27.3 | 26.5 | 25.3 |
| (MPa) | | | | |
| Elongation at | 5.47 | 17.11 | 13.29 | 22.57 |
| Break (%) | | | | |
| Elongation at | 2.72 | 0.80 | 5.00 | 3.56 |
| Yield (%) | | | | |

* All ingredient percentages are on a weight basis.

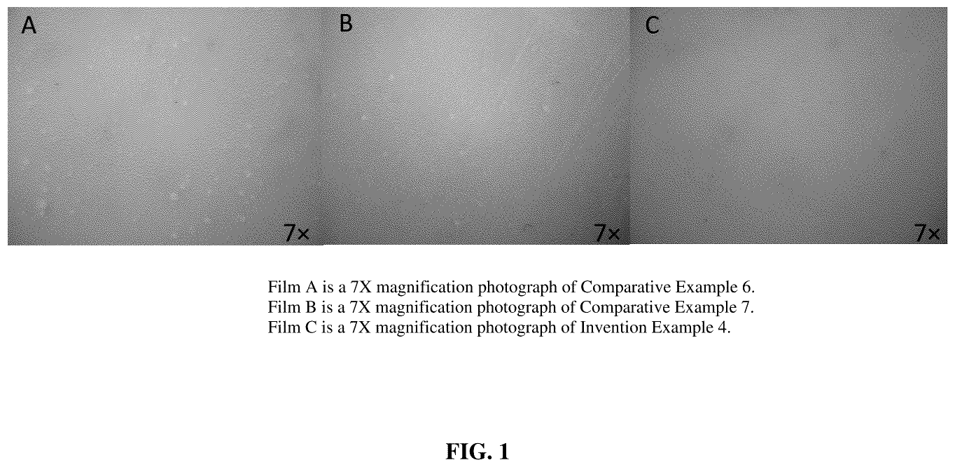

As is demonstrated by the results shown in Table 1, while the invention examples showed similar flame retardancy and physical properties when compared with the comparative flame retardant examples, as evidenced by FIG. 1, resin blends containing the flame retardants of the invention do not contain particles which cause unwanted occlusions during extrusion, unlike the prior art flame retarded resin blends.

To demonstrate that meltable aminoethyl piperazine pyro/polyphosphates in accordance with the invention have advantages compared to non-meltable commercial products, samples of flame retardant containing PP resins (Examples 4, 6, and 7 of Table 1) were pressed into 0.15 mm thin films and examined using a microscope. Particles and agglomerations in the extrusion melt can impact the extrusion process. The 7× magnified image of the comparison melamine based ammonium polyphosphate commercial formulation (Film A of FIG. 1) and the piperazine pyrophosphate and melamine pyrophosphate commercial formulation (Film B of FIG. 1) shows numerous particles and agglomerations. However, the 7× magnified Film C of FIG. 1 clearly shows that meltable flame retardant formulations in accordance with the invention formed a smooth blend, without any particles or agglomerations that could impact extrusion.

While the above description contains many specifics, these specifics should not be construed as limitations of the invention, but merely as exemplifications of preferred embodiments thereof. Those skilled in the art will envision many other embodiments within the scope and spirit of the invention as defined by the claims appended hereto.

1. A flame retardant compound having the general formula (I): 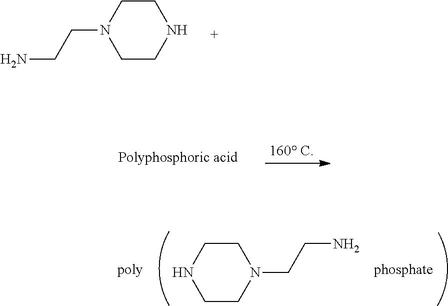
wherein x≥2; y is from 2 to 6; and z≥1; and x≥z, provided that when z is 1, x is ≥2.

2. The flame retardant compound of claim 1, wherein x is from 2 to 6 and z is from 1 to 6.

3. The flame retardant compound of claim 1, wherein x is from 2 to 4, y is from 2 to 4, and z is from 1 to 4.

4. The flame retardant compound of claim 1, wherein y is 2.

5. The flame retardant compound of claim 1, wherein x is 2.

6. The flame retardant compound of claim 1, wherein x and y are 2 and z is 1.

7. A flame retarded thermoplastic resin composition comprising a thermoplastic polymer and a flame retardant compound having the general formula (I): 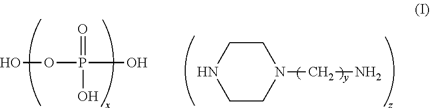
wherein x≥2; y is from 2 to 6; z≥1; and x≥z, provided that when z is 1, x is ≥2 and mixtures thereof.

8. The flame retarded thermoplastic resin composition of claim 7, wherein the thermoplastic polymer comprises a polyolefin.

9. The flame retarded thermoplastic resin composition of claim 7, further comprising at least one of ammonium polyphosphate, ammonium pyrophosphate, piperazine polyphosphate, piperazine pyrophosphate. melamine polyphosphate, ethylenediamine phosphate, or melamine pyrophosphate.

10. The flame retarded thermoplastic resin composition of claim 8, wherein the polyolefin is polypropylene.

11. The flame retarded thermoplastic resin composition of claim 8, wherein the flame retardant compound is selected from the group consisting of aminoethyl piperazine pyrophosphate, aminoethyl piperazine polyphosphate and mixtures thereof.

12. The flame retarded thermoplastic resin composition of claim 7, wherein the resin composition substantially lacks observable particles of the flame retardant compound.

13. The flame retarded thermoplastic resin composition of claim 7, wherein the flame retardant compound is present in at least 10% by weight of the resin composition.

14. A metal wire or cable covered with a resin, said resin comprising a polyolefin resin and a flame retardant compound having the general formula (I): 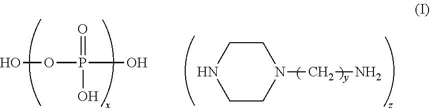
wherein x≥2; y is from 2 to 6; z≥1; and x≥z, provided that when z is 1, x is ≥2, and mixtures thereof.

15. A plastic molded component adapted for use in electrical or electronic equipment, said component comprising at least one thermoplastic resin and a flame retardant compound having the general formula (I): 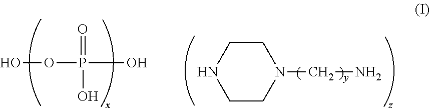
wherein x≥2; y is from 2 to 6; z≥1; and x≥z, provided that when z is 1, x is ≥2, and mixtures thereof.

16. A method of preparing a flame retardant compound having the general formula (I): 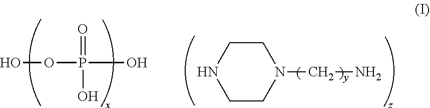
wherein x≥2; y is from 2 to 6; z≥1; and x≥z, provided that when z is 1, x is ≥2, comprising the step of reacting aminoalkyl piperazine with polyphosphoric acid.

17. The method of claim 16, wherein the process comprises reacting a mixture of piperazine and aminoalkyl piperazine with phosphoric acid and then heating the reaction product therefrom.

18. The plastic molded component adapted for use in electrical or electronic equipment of claim 15, wherein said component comprises a casing for said equipment.
